(19) United States
(12) Patent Application Publication
(10) Pub. No.: US 2025/0246318 A1
(43) Pub. Date: Jul. 31, 2025

(54) BIOMEDICAL KNOWLEDGE GRAPH

(71) Applicant: Roche Molecular Systems, Inc., Pleasanton, CA (US)

(72) Inventors: Chaohui GUO, Basel (CH); Vishakha SHARMA, Pleasanton, CA (US); Antoaneta VLADIMIROVA, Pleasanton, CA (US)

(21) Appl. No.: 19/037,080

(22) Filed: Jan. 25, 2025

Related U.S. Application Data
(63) Continuation-in-part of application No. PCT/US2023/070822, filed on Jul. 24, 2023.
(60) Provisional application No. 63/369,470, filed on Jul. 26, 2022.
(60) Provisional application No. 63/379,010, filed on Oct. 11, 2022.
(60) Provisional application No. 63/563,074, filed on Mar. 8, 2024.

(51) Int. Cl. G16H 50/70; G06F 16/2452; G06N 5/022

(52) U.S. Cl. CPC G16H 50/70; G06F 16/24522; G06N 5/022

(57) ABSTRACT

A biomedical knowledge graph system includes a computer database of records comprising nodes of biomedical entities and connections between the entities representing biomedical relationships. One or more processors are configured to extract data from a plurality of data sources and determine biomedical entities and relationships between the entities based on analyzing the data, including searching for predetermined identifiers or patterns in the data. Based on the determined biomedical entities, each biomedical entity is assigned to a cluster of biomedical entity types and a context is identified for each of the entities. Based on the identified context and type of the biomedical entity, records of nodes and connections between nodes are incorporated into the knowledge graph, the nodes representing biomedical entities and the connections representing biomedical relationships between the entities structured according to the predefined schema.

### CROSS-REFERENCE TO RELATED APPLICATIONS

This application is a bypass continuation-in-part of International Application No. PCT/US2023/070822, filed on Jul. 24, 2023, which claims the benefit of and the priority to U.S. Provisional Application No. 63/369,470 filed on Jul. 26, 2022 and U.S. Provisional Application No. 63/379,010 filed on Oct. 11, 2022, and further claims the benefit and the priority to U.S. Provisional Application 63/563,074 filed on Mar. 8, 2024, which are hereby incorporated by reference in their entireties for all purposes.

### BACKGROUND

Rapidly obtaining the most updated and accurate information for performing clinical care and biomedical research is critical. Vast amounts of information is distributed through a wide variety of resources including published guidelines, periodicals, clinical studies, and online medical compendiums. Searching through these materials and extracting relevant updated information can be cumbersome and time consuming.

Finding updated information often involves use of generalized search tools with key words or phrases, often yielding inconsistent results and non-relevant material (e.g., non-medical or outdated material). Some compilations of materials may not be updated with the most recent and accurate information. Updating them often relies on manual review or verification by skilled (or unskilled) personnel, making them potentially unreliable and/or outdated.

There is thus a need for methods of obtaining relevant and updated biomedical information in an efficient and timely manner.

### SUMMARY

Aspects of the disclosure provide systems and methods for generating and operating a biomedical knowledge graph from a plurality of disparate sources in a context-based graphical structure. Queries for biomedical information may be adapted and used to search the knowledge graphs in a highly efficient and targeted manner for obtaining biomedical information. The sources of information may include periodicals, clinical trial results, biomedical compendiums, news articles, and other sources, including online sources.

A biomedical knowledge graph is generated by first accessing source material and extracting it (e.g., using a natural language processor (NLP)), based on which medical data entities and relationships between the entities are established. Entities may include certain diseases, therapies, and tests, for example, and a relationship can be defined by an association between entities. A relationship, for example, may include a test entity used diagnose a particular disease entity or a therapy to treat the disease. Multiple relationships between multiple entities may be extracted. In some embodiments, these entities and relationships are established and/or verified utilizing machine-learning analysis for parsing and extracting information from multiple sources and validating their relevance and accuracy based on the analysis.

In some embodiments, after obtaining data from one or more sources, predetermined identifiers or patterns in the data are identified such as through the use of an identifier index or named entity resolution (NER) module to determine entities and biomedical entity types. Clusters of biomedical entity types (or themes) are established (e.g., by machine learning) for particular types of biomedical entities to which they are assigned. Context types for the biomedical entities are identified based on the assigned cluster and/or by analyzing aspects of the data from which the entities were extracted. Based on the identified context type and entity type of the biomedical data, an entry or record is added into a biomedical knowledge graph according to a predefined schema. A context may include an identification of a biomarker associated with a disease, a gene sequence, and/or a hypothesis within a medical publication, for example.

In some embodiments, queries for biomedical information or answers from the biomedical knowledge graph are facilitated through a query engine configured to interpret structured or unstructured queries (e.g., natural language questions, standard query language-based queries). In some embodiments, the query is converted into a structured query form based on the predefined schema of the biomedical knowledge graph for rapid access and retrieval of relevant information. Conversion may include use of a natural language processor (e.g., named entity resolution) to correlate portions of a query with entities and entity types of the biomedical knowledge graph, along with the relationships inquired about.

Query results or answers can include a traversable graph of entities and their relationships pertaining to the query. Results can also include tabulated results with corresponding information about each particular result and its elements (e.g., treatment for a particular disease and success rate). Links or summaries of the sources of the information (e.g., particular clinical trials) may be embedded or accessible with generated results. Reports including statistical analysis of the results may also be generated.

### BRIEF DESCRIPTION OF THE DRAWINGS

Various objects and advantages of the disclosure will be apparent upon consideration of the following detailed description, taken in conjunction with the accompanying drawings, in which like reference characters refer to like parts throughout, and in which:

FIG. 1 is an illustrative diagram of processes for constructing a biomedical knowledge graph from disparate data sources according to some embodiments.

FIG. 2A is an illustrative diagram of a process for constructing a biomedical knowledge graph from a generalized knowledge graph according to some embodiments.

FIG. 2B is an exemplary list of biomedical entity types from a generalized knowledge graph.

FIG. 2C is an exemplary list of relationship types between entities extracted from a generalized knowledge graph according to some embodiments.

FIG. 3 is an illustrative diagram of a process for extracting biomedical data from a general data source according to some embodiments.

FIG. 4 is an illustrative diagram of a process for extracting biomedical data from clinical trial sources according to some embodiments.

FIG. 5 is a representative schema for a biomedical knowledge graph according to some embodiments.

FIG. 6 is a process for executing a query of a biomedical knowledge graph according to some embodiments.

FIG. 7A is a graphical user interface for building a query according to some embodiments.

FIG. 7B is executable query code generated from the query built from the interface of FIG. 7A.

FIG. 8A is query output from a biomedical knowledge graph in a navigable knowledge graph representation according to some embodiments.

FIG. 8B is a query output from a biomedical knowledge graph in a tabular representation according to some embodiments.

FIG. 9A is a trend output from a biomedical knowledge graph according to some embodiments.

FIG. 9B is a graphical representation of predicted responses for a particular disease or condition in response to particular therapeutic compounds according to some embodiments.

FIG. 10A is an exemplary process for training and utilizing a machine learning system for predicting alternative paths between therapeutic indicators using a biomedical knowledge graph according to some embodiments.

FIG. 10B is an exemplary output of paths between a sequence variant to drug found within a biomedical knowledge graph according to some embodiments.

FIG. 10C is an exemplary output of an alternative path based on the machine learning process of FIG. 10A.

FIG. 11 is an illustrative flow diagram of processes for identifying relationships between entities in a biomedical knowledge graph according to some embodiments.

FIG. 12 is an illustrative flow diagram of processes for identifying a therapeutic candidate from a biomedical knowledge graph based on a particular sequence variant according to some embodiments.

FIG. 13 is an exemplary output of candidate therapeutics with predicted response characteristics generated from a biomedical knowledge graph in relation to particular sequence variants according to some embodiments.

FIG. 14 is an illustrative diagram of computing devices and processing components of a system for generating and utilizing biomedical knowledge graphs according to some embodiments.

### DETAILED DESCRIPTION

Aspects of the disclosure include methods and systems for generating and querying biomedical knowledge graphs constructed from numerous disparate data resources. The resources from which the biomedical knowledge graphs are generated include existing generalized knowledge graphs and biomedical-specific resources such as, for example, clinical trial data and other published materials. Some embodiments include systems and methods for extracting data from these resources and building/updating/augmenting biomedical knowledge graphs according to a uniform schema using methods adapted to the type and form of resource from which the data is extracted. Some embodiments include methods for querying the biomedical knowledge graphs in order to obtain results to reflect optimally up-to-date, relevant, and accurate medical information.

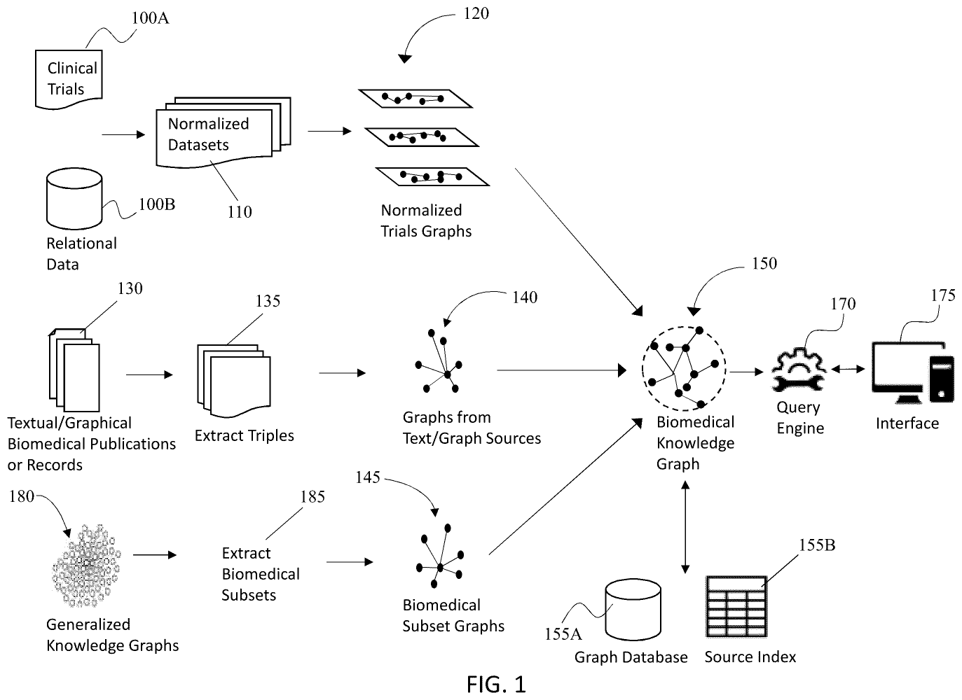

FIG. 1 is an illustrative diagram of processes for constructing a biomedical knowledge graph from disparate data sources according to some embodiments. A biomedical knowledge graph 170 is constructed by extracting from existing biomedical data that may include a knowledge graph 150 of generalized data (e.g., including but not limited to biomedical data), biomedical publications 130 such as journal articles, and clinical trials data 100A (e.g., summarized from a publication) and clinical trials relational data 100B (e.g., raw data records).

In some embodiments, a biomedical knowledge graph represents nodes of biomedical entities connected by contextual relationships between the entities. For example, a node represent a disease, condition, therapy, molecule, etc . . . , and a relationship may be a treatment or therapy with a molecule for a disease or condition, for example. These nodes and relationships may be represented according to a pre-defined schema or structure.

A generalized (or generic) knowledge graph 180 (e.g., which can include any graph-structured representation of data) is accessed and parsed at 185 to identify portions of the knowledge graph related to biomedical information. The identified portions are represented as biomedical subset graphs 145. For example, a generalized knowledge graph may include Wikidata (https://www.wikidata.org/wiki/Wikidata: Main_Page), DBpedia (https://www.dbpedia.org/), and/or many others.

In order to extract biomedical data from a generalized knowledge graph 150, some data is identified by particular identifiers or predicates (e.g., alphanumeric IDs) already known or classified as representing biomedical information (e.g., particular diseases, therapeutic compounds, and/or types thereof). In some embodiments, data is identified as biomedical information utilizing a named entity resolution (NER) and/or machine learning component such as further described herein.

In some embodiments, the biomedical subsets 185 are (re)structured utilizing a predefined schema and/or ontology consistent across the biomedical knowledge graph 150. In some embodiments, subsets 145 of a generalized knowledge graph form the basis of our biomedical knowledge graph 170, which may then be augmented using data extracted from other sources such as biomedical publications 130, clinical trial data 100A and 100B, and information/updates of generalized knowledge graphs such as further described herein.

In order to extract biomedical data from textual/graphical biomedical publications or records 130 that are typically unstructured, the text/graphics of the publications or records 130 are parsed such as utilizing a NLP/NER, image processing module, and/or machine learning system. Triples (or other structured forms) of the extracted data are generated at 135. Some examples of entity resolution methods include NCBI Gene and Spark NLP. Other examples of deriving biomedical relationships from text include Percha B, Altman R B. “A global network of biomedical relationships derived from text.” Bioinformatics. 2018 Aug. 1; 34(15): 2614-2624. The extracted/structured biomedical data is incorporated into a (subset) biomedical knowledge graph 140 consistent with the predefined schema/ontology of the biomedical knowledge graph 170. Nodes and relationships of the biomedical knowledge graph may be stored within a computer database 155A and indexed within source index 155B.

In some embodiments, data for the knowledge graph 170 is obtained or enhanced from clinical trials and/or other trial, medical images, diagnostic test data, and/or other historical or analytical data sources 100A and 100B (e.g., FDA submissions). In some embodiments, clinical trials data may be in textual form in 100A (e.g., from a report or journal publication) and/or in a data format in 100B (e.g., a relational table of results). Once the data is extracted, it can be collated into normalized datasets 110.

The normalized data sets 110 are then translated/converted into normalized

knowledge graphs 120. The normalized knowledge graphs 120 are then used to augment biomedical knowledge graph 170. For example, the relationship and correlation between entities in the knowledge graph (e.g., a compound and treatment of a condition) may be established, reinforced, or discounted by the data.

In some embodiments, these various data sources are periodically monitored (e.g., “scraped”) to determine if new/updated data is available for augmenting/updating biomedical knowledge graph 170. For example, data that is periodically identified in these data sources can be compared to the entities and relationships in the knowledge graph 170 to determine if an update is needed. In some embodiments, information about the original source (e.g., website URL, citation) from which the data originated is stored with the biomedical knowledge graph and made accessible in results of queries performed on the knowledge graph that contain entities and relationships pertaining to the search results.

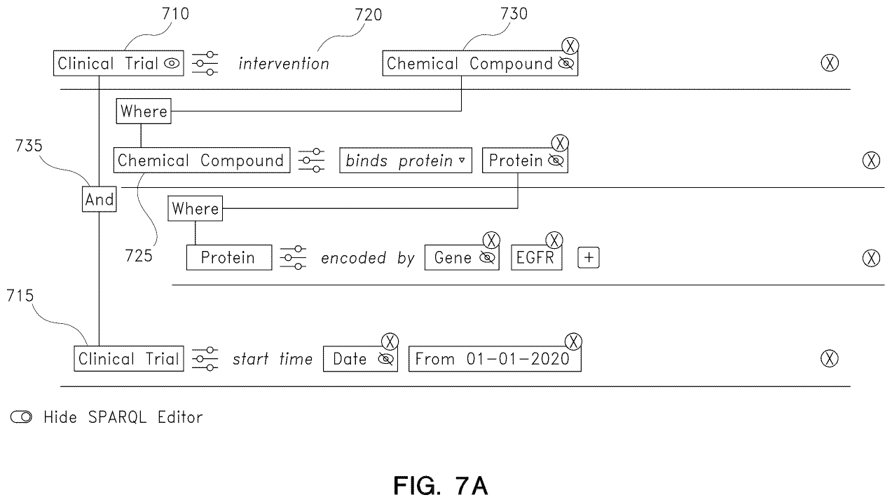

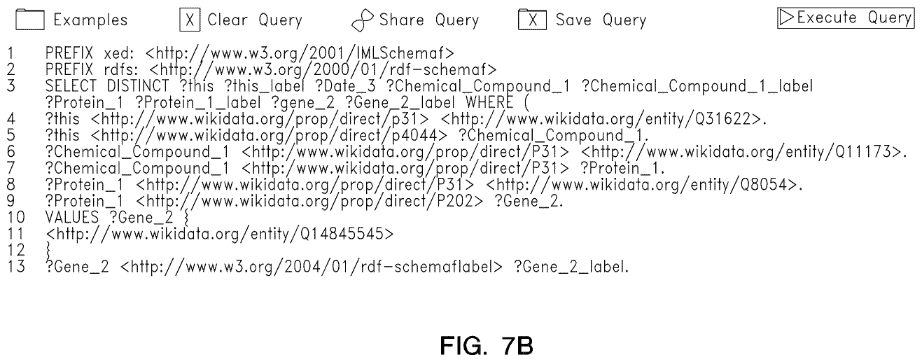

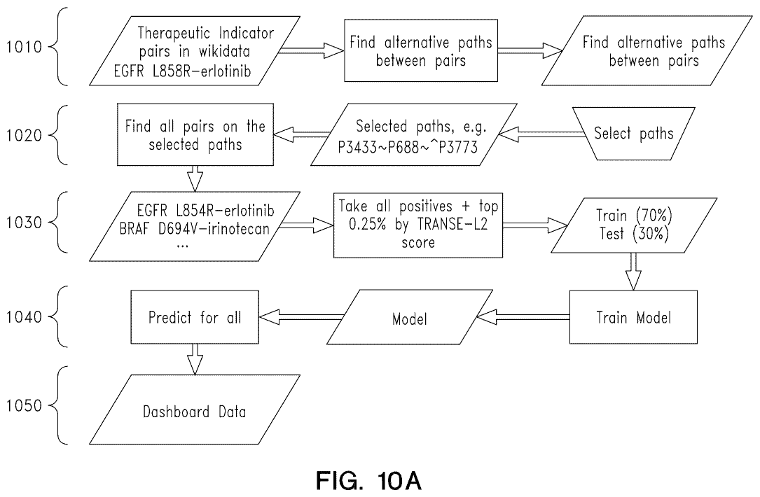

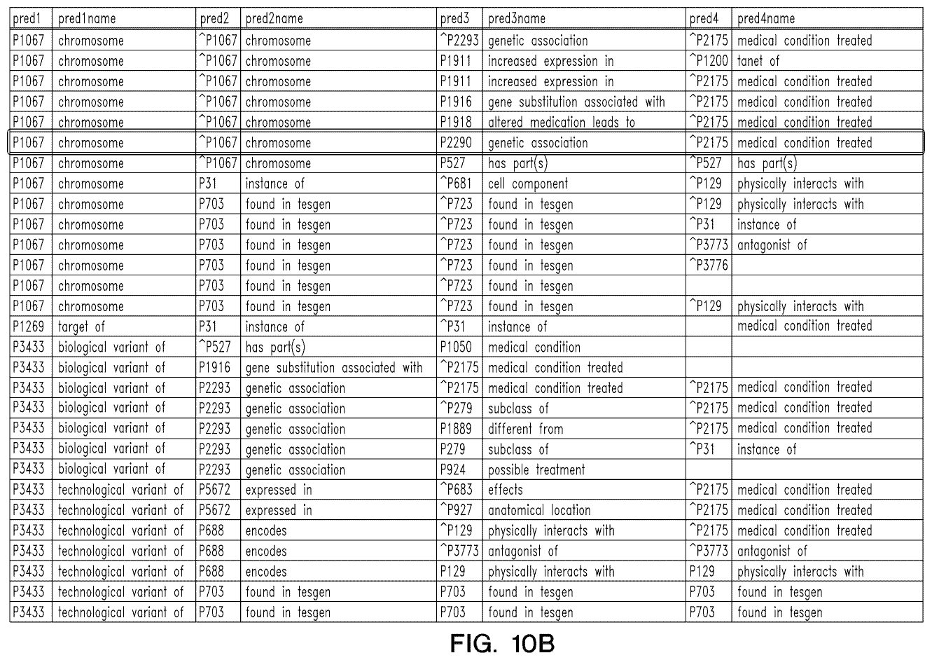

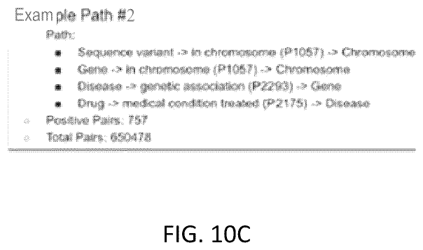

Once biomedical knowledge graph 170 has been generated, queries can be performed utilizing the knowledge graph to rapidly obtain requested biomedical information and/or to perform analysis (e.g., machine learning model generation as described in reference to FIGS. 10A-10C) for predicting biomedical relationships and/or reporting trends. A query engine 175 may be utilized to perform queries such as received through an external interface (e.g., a GUI as further described in reference to FIGS. 7A and 7B). Query engine 175 is configured to perform queries particularly adapted to biomedical knowledge graphs as described further herein.

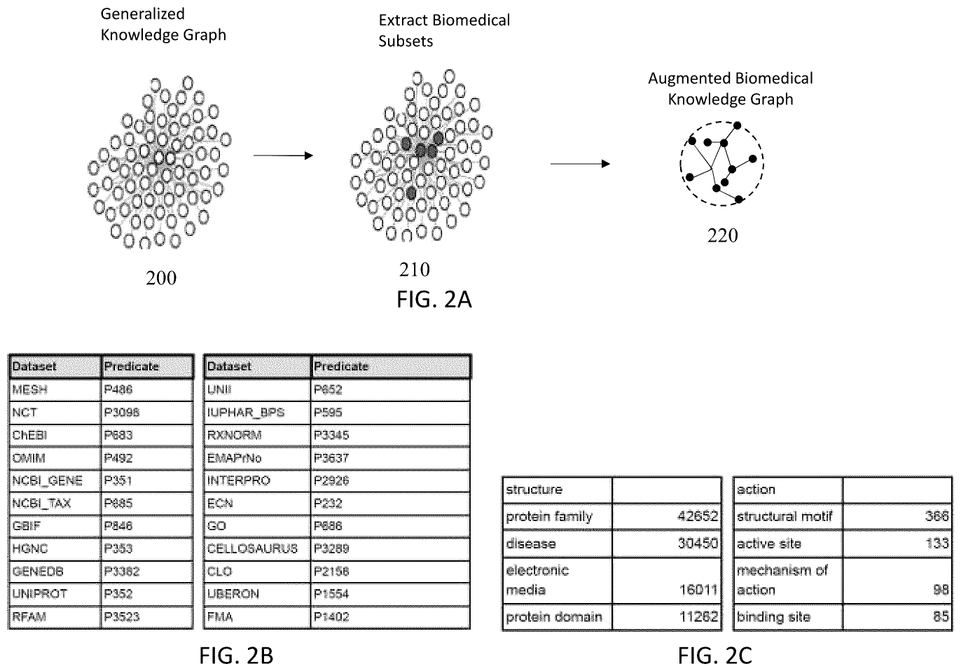

FIG. 2A is an illustrative diagram of a process for constructing a biomedical knowledge graph from a generalized knowledge graph. A generalized knowledge graph 200 may include information regarding a wide array of data types and sources. For example, WikiData provides a knowledge graph with a large breadth of data pertaining to history, social topics, politics, and science, including biomedical information. Some portions of the generalized knowledge graph 210 may be identified as pertaining to biomedical information. Identification may be performed utilizing identifiers or predicates associated with the generalized knowledge graph that identify entities and relationships by type or context (e.g., particular disease, treatment, or a type of biomedical information).

FIG. 2B is an exemplary list of biomedical entity types from a generalized knowledge graph and FIG. 2C is an exemplary list of relationship types between entities extracted from a generalized knowledge graph according to some embodiments. As indicated above, these types and/or relationships may be explicitly identified and represented as biomedical-related. In some embodiments, the types or contexts (and/or the data itself) of a generalized knowledge graph are analyzed (e.g., using NLP/NER) and classified as biomedical information (or not) based on the analysis. In some embodiments, the relationships include one or more of positively regulating or negatively regulating biological factors. In some embodiments, the relationships include at least one of an action via a receptor, indirect alteration of an effect of an endogenous agonist, the inhibition of transport processes, enzyme inhibition, enzymatic action or activation of enzymatic activity, chelation, osmosis, and/or anesthesia.

After portions of the generalized knowledge graph are identified as biomedical information, these portions are incorporated into a biomedical knowledge graph 220. The incorporated portions may be used as a basis or foundation for a biomedical knowledge graph and/or used to augment an existing biomedical knowledge graph 220. In some embodiments, the schema of the biomedical knowledge graph 220 maintains the original schema of the generalized knowledge graph 200 or, in some embodiments, incorporated data is adapted/converted to another predetermined schema. For example, information from the original generalized knowledge graph 200 may not have utilized a particular predicate or identifier for a type of biomedical type, relationship, and/or context and, based on analyzing the data, it is classified with a particular biomedical identifier and/or context according to a schema/index of the biomedical knowledge graph 220.

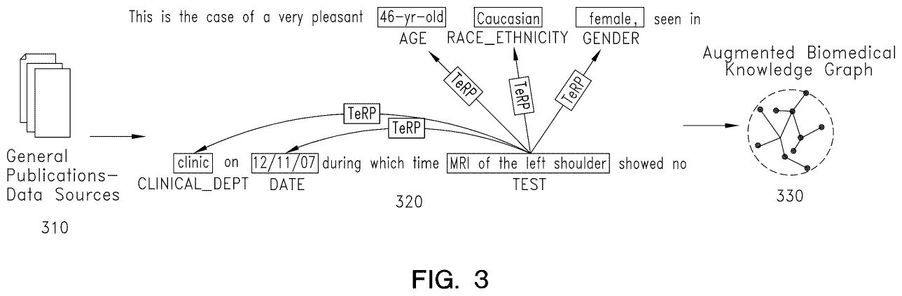

FIG. 3 is an illustrative diagram of a process for extracting biomedical data from a general data source according to some embodiments. General data source 310 may include periodicals, website content, medical records or reports, and/or other sources in which the data may not be structured in a predetermined way. This general data may be analyzed at 320 and extracted such as by utilizing a NLP (e.g., NER) to identify biomedical information. The analyzing and extraction may include identifying and extracting triples (e.g., entities and relationships) from one or more sentences of unstructured text. Entities, relationships, and contexts are based on the analyzed data. The extracted entities, relationships, and/or contexts are then incorporated into the biomedical knowledge graph 330. Incorporation can include adding, updating, or removing portions of the biomedical knowledge graph 330 based on analyzing and/or comparing the extracted data with data in the biomedical knowledge graph 330.

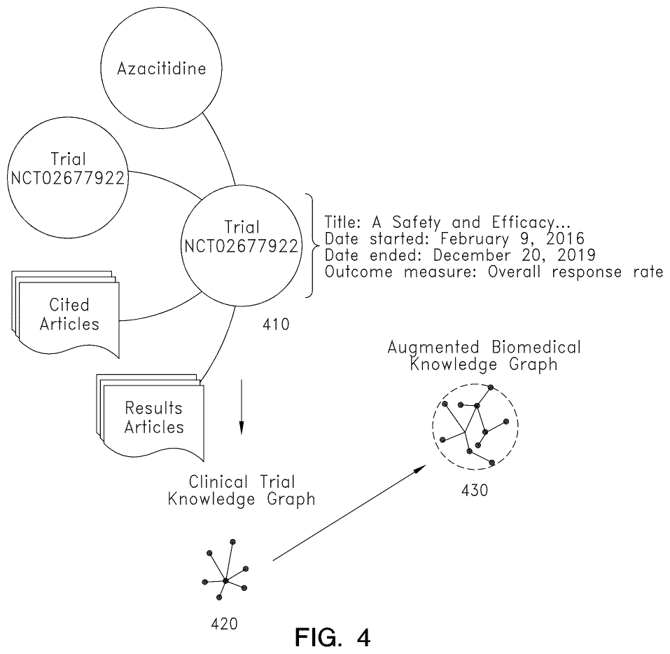

FIG. 4 is an illustrative diagram of a process for extracting biomedical data from clinical trial sources according to some embodiments. Clinical trials sources 410 provide information about clinical trials including, for example, government reporting sites, academic centers, journal publications, internal/proprietary testing records, and other sources. In some cases, the format of the trials data may include summaries, statistics, and/or raw data. Information based on summaries and statistics may be obtained in a similar way as that described in reference to general data sources and translated into entities and relationships for a clinical trials graph 420. Raw data may also be extracted, after which statistics are calculated on the raw data and used, for example, to establish entities and relationships for the clinical trials knowledge graph 420.

Clinical trials graph 420 is then incorporated into (e.g., adding, augmenting) biomedical knowledge graph 430. Certain entities and relationships, for example, can be added, removed, or updated based on comparing them with those of biomedical knowledge graph 430.

In some embodiments, a weight is determined and associated with clinical trials data 410 before it is incorporated. For example, the strength of a relationship may be determined based on a determined reliability or accuracy of the associated data and associated with it in the clinical trials graph 420. The weight or strength of identified relationships may then, for example, be represented in results or analysis (e.g., trends) generated in response to queries (e.g., by query engine 175 of FIG. 1).

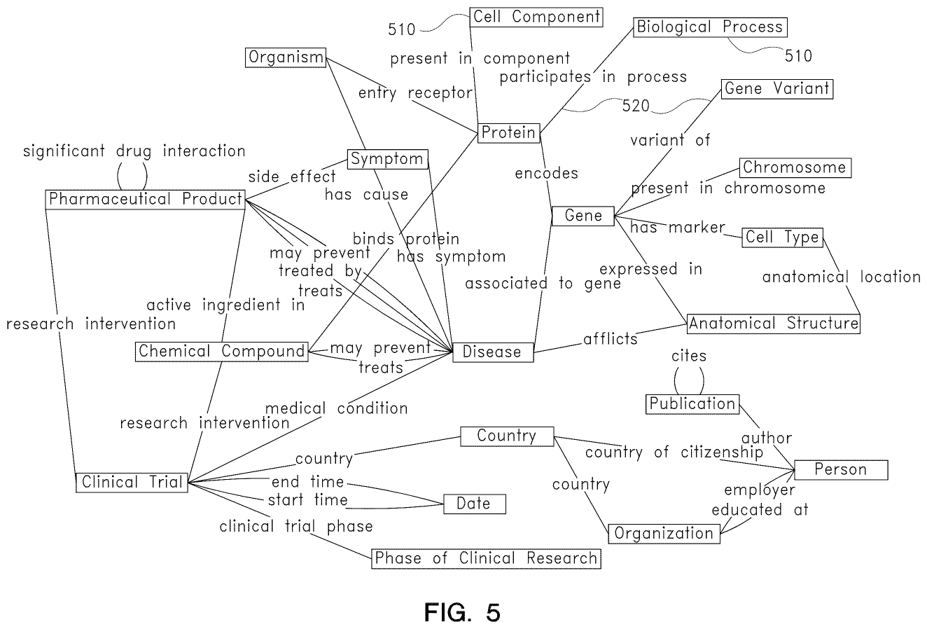

FIG. 5 is a representative schema for a biomedical knowledge graph according to some embodiments. Numerous exemplary biomedical entity types 510 and relationship types 520 are represented. When populating a biomedical knowledge graph, elements or entities of extracted data (e.g., a disease, gene) are correlated with one or more biomedical entity types, and recorded as nodes by their type(s). Relationships with other entities are identified by relationship type (e.g., gene expressions) and recorded as connections between nodes. In some embodiments, relationships are identified based on determining a particular context in which the data is presented (e.g., type of clinical trial, diseased or healthy patients, stage of cancer, geographic location) and can be enhanced with machine learning techniques. In some embodiments, entities are classified as a gene, sequence, anatomic structure, chemical substance, disease, and/or phenotypic feature.

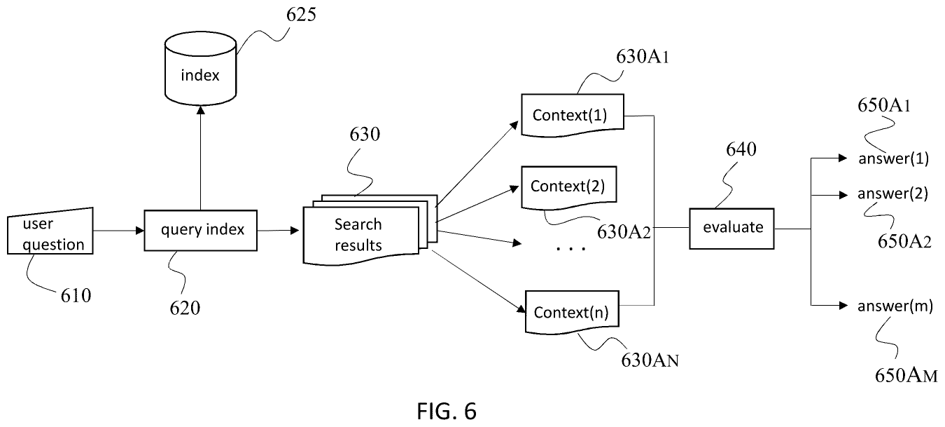

FIG. 6 is a process for executing a query of a biomedical knowledge graph according to some embodiments. At 610, a structured or unstructured query (or question) is received (e.g., “what are the top treatments for squamous-cell carcinoma of the lung?”). In some embodiments, the query is translated and structured into a format (if necessary) for performing a lookup utilizing a query index search 620 and index of terms 625. At 630, results from the search are generated and may include results pertaining to many possible contexts 630A1, 630A2, . . . , 630AN (e.g., stage of cancer). At 640, the result(s) are analyzed and verified for conformity with particular quality criteria (e.g., utilizing a generative adversarial network (GAN)). Based on the analyzing, conforming/verified results 650A, . . . ,650M are presented as answers answer(1), . . . , answer(m). The answers may be transmitted to a recipient (e.g., across a computer network) and displayed to a user (e.g., in a graphical user interface).

FIG. 7A is a graphical user interface for building a query according to some embodiments. The interface provides fields for entering search terms (e.g., at 710, 715, and 730), relationships between the terms (e.g., at 720), and qualifiers (e.g., at 735) pertaining to the terms and relationships for building a query. In some embodiments, fields for terms, relationships, and/or qualifiers are configured with pre-populated and selectable entries or options.

FIG. 7B is executable query code generated from the query built from the interface of FIG. 7A. Based on the entries and/or selections entered in the interface of FIG. 7A, query code for executing a biomedical graph query is generated such as illustrated. The code may be generated according to a particular query format or language such as those known to one of ordinary skill in the art (e.g., SQL, GraphQL, XQuery, JSONiq, etc. ).

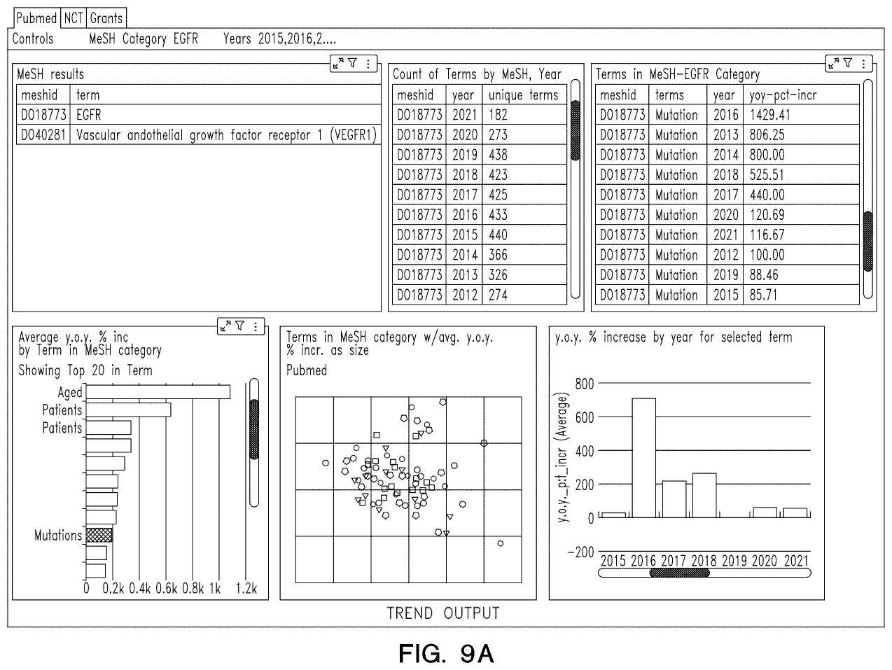

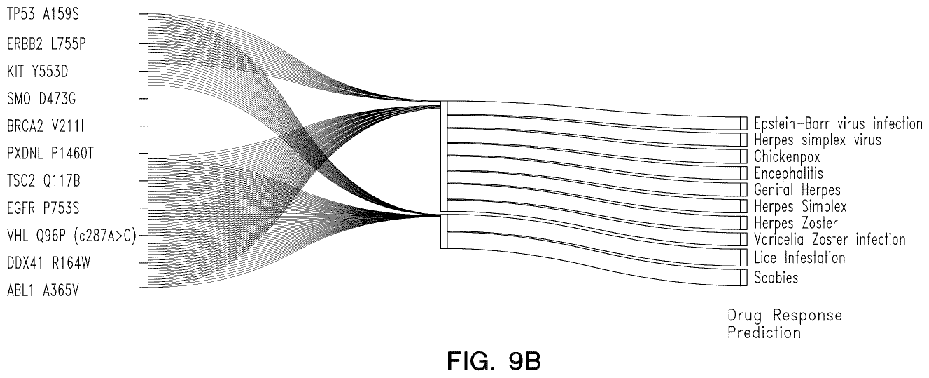

A query may be received in textual form. The query is then parsed (e.g., utilizing an NLP/NER) and analyzed to identify biomedical entities and relationships to be searched in connection with the query. The identified entities and relationships can be identified/correlated with respect to those of the biomedical knowledge graph. The biomedical knowledge graph is searched (e.g., by way of an index of terms) for the identified entities relating to the queried relationships (e.g., a cluster of relationships) between the entities. The search may be narrowed to relationships between the entities that best correspond to the query (e.g., a context identified/resolved from the query text). Results may be further evaluated for conformance with expected standards (e.g., based on a trained adversarial network). The results may be translated/converted into a particular format (e.g., sentence form) and/or used to generate statistical and/or trend analysis (e.g., as illustrated in FIGS. 9A and 9B).

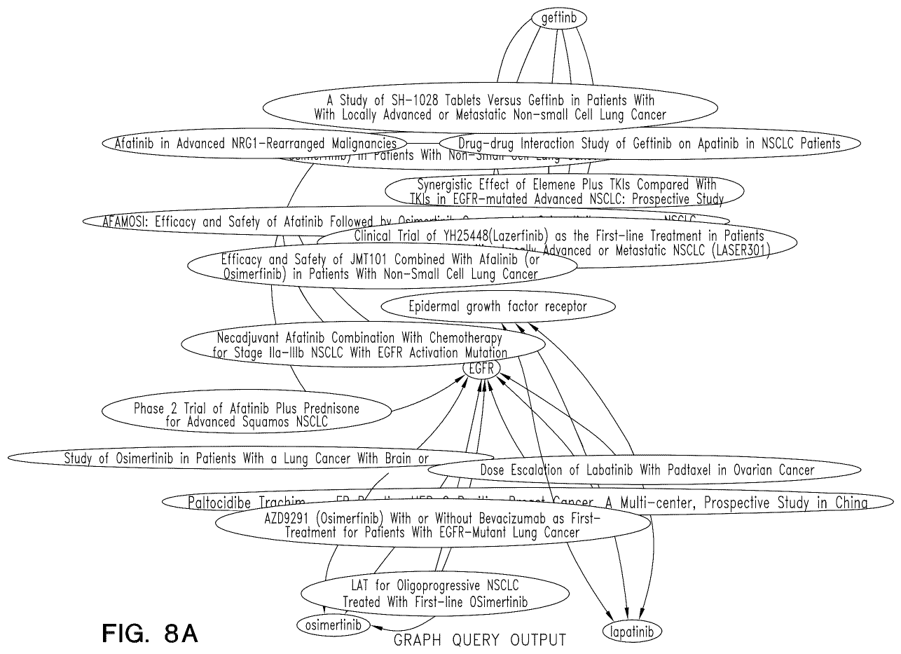

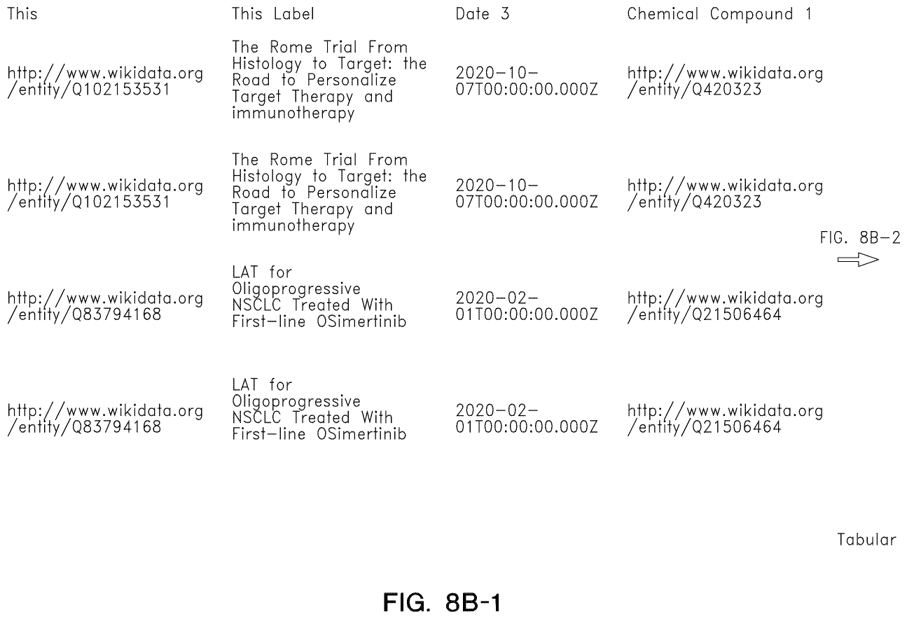

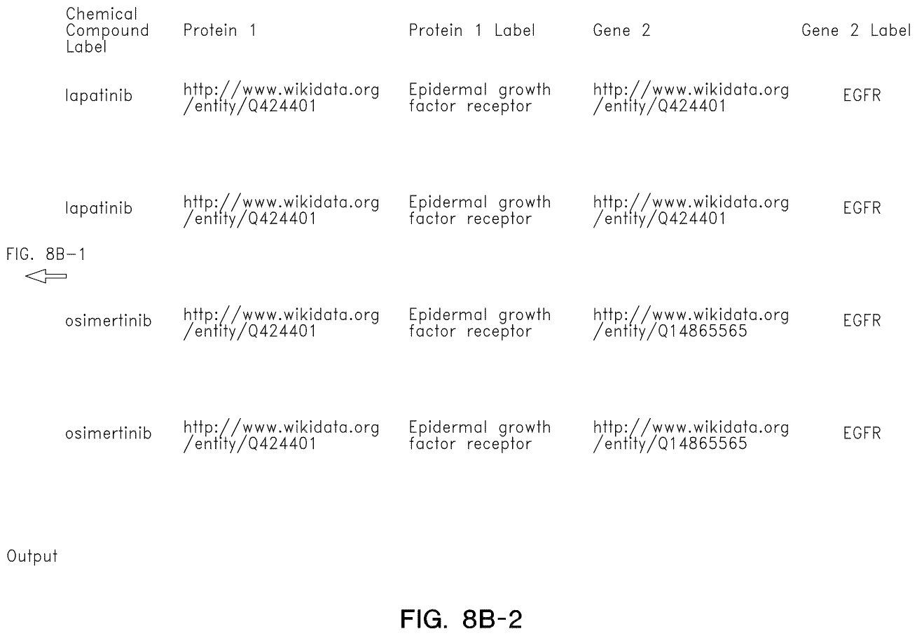

FIG. 8A is query output from a biomedical knowledge graph in a navigable knowledge graph format according to some embodiments. Numerous results may be generated in response to some queries, particularly broad queries. In order to permit easier navigation of these results, they are generated in a knowledge graph format including the potentially numerous result entities and relationships between them. Because the queried source is originally in a knowledge graph form, generating results in similar form is relatively fast and efficient. In some embodiments, links or citations to original sources (e.g., publications, clinical trial data, images) may be accessible or navigable within a graph-based interface as they pertain to results/relationships identified from the queried biomedical knowledge graph.

FIG. 8B is a query output from a biomedical knowledge graph in a tabular representation according to some embodiments. A set of results may also be presented in a table or spreadsheet form such as in rows and columns of various result fields. That way, the results themselves may be more easily organized, analyzed, and/or searched. For example, charts or plots may be generated based on tabulated results such as illustrated in FIGS. 9A and 9B.

FIG. 9A is a trend output from a biomedical knowledge graph according to some embodiments. Based on analyzing results of one or more queries of a biomedical knowledge graph, trends in the result data are generated and presented. For example, a chart of the relative proportions of patients successfully responding to particular therapies can be presented with respect to each other.

FIG. 9B is a graphical representation of predicted responses for a particular disease or condition in response to particular therapeutic compounds. Predicted responses may be based on machine learning models trained with data accessible in a biomedical knowledge graph. For example, particular compounds may be predicted to access therapeutic pathways or provide outcomes based on historical use of the compound for the treatment of other diseases, similarities in chemical structure to other compounds with positive outcomes, and/or related treatments of similar conditions.

FIG. 10A is an exemplary process for training and utilizing a machine learning system for predicting alternative paths between therapeutic indicators using a biomedical knowledge graph according to some embodiments. FIG. 10B is an exemplary output of paths between a sequence variant to drug found within a biomedical knowledge graph according to some embodiments. FIG. 10C is an exemplary output of an alternative path based on the machine learning process of FIG. 10A. The aspects 1010 of the process include obtaining therapeutic indicator pairs from the biomedical knowledge graph and determining all possible alternative paths between the pairs. At 1020, paths between the pairs are selected (e.g., based on a type of path) and the biomedical knowledge graph data is further searched to identify all pairs on the selected paths. At 1030, criteria is used to narrow the identified pairs to those most likely to have a positive therapeutic impact. At 1040, a portion (e.g., 70%) of the narrowed set is used to train a machine learning model to generate responses to queries, generate analytics, and/or clinical predictions (e.g., predicting effective therapies, alternative paths), for example, while another portion (e.g., 30%) is used to test hypothesis generated by the model.

The above-described embodiments of the present disclosure are presented for purposes of illustration and not of limitation, and the present disclosure is limited only by the claims which follow. Furthermore, it should be noted that the features and limitations described in any one embodiment may be applied to any other embodiment herein, and flowcharts or examples relating to one embodiment may be combined with any other embodiment in a suitable manner, done in different orders, or done in parallel. In addition, the systems and methods described herein may be performed in real time. It should also be noted, the systems and/or methods described above may be applied to, or used in accordance with, other systems and/or methods.

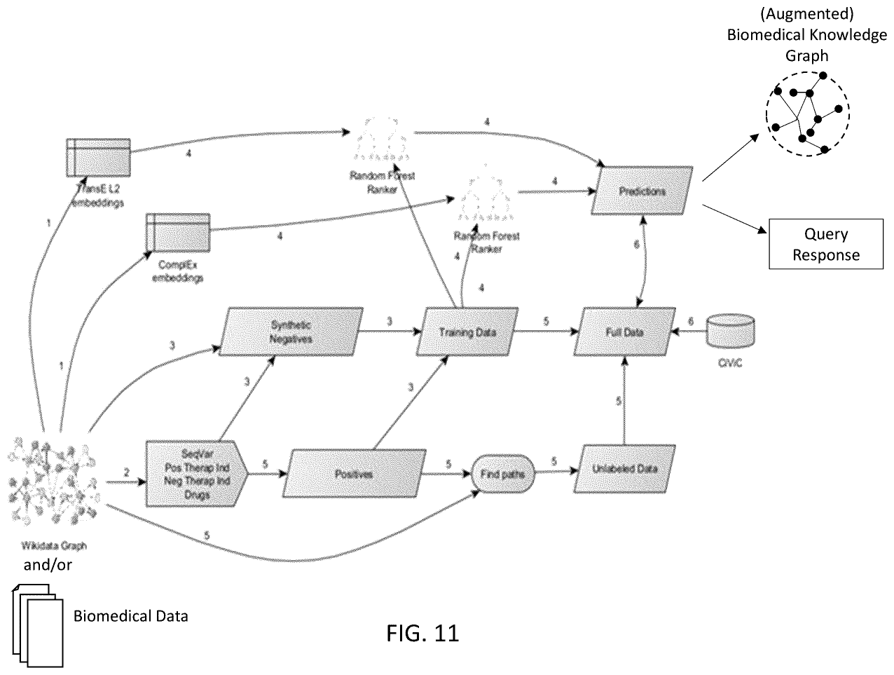

FIG. 11 is an illustrative flow diagram of processes for identifying relationships between entities in a biomedical knowledge graph according to some embodiments. In some embodiments, predicted relationships are generated based on translations of established relationships within biomedical data, including, for example, previously established relationships within a knowledge graph. The predictions may include indicators (e.g. probability) of likely positive therapeutic impact, negative therapeutic impact, or no therapeutic impact.

A machine learning model is trained to identify relationships within the biomedical data and/or knowledge graph based on a dissimilarity measure between related entities. Biomedical entities, entity types, and entity relationships are determined in the data by searching for predetermined identifiers or patterns in the data and based on output from the machine learning model in response to an input of relationships and entities.

In some embodiments, the dissimilarity measure includes applying a margin-based ranking criterion using a margin hyperparameter over established relationships and applying a loss function to triplets extracted from the established relationships. For example, Bordes, et al. describe methods for translating embeddings for modeling multi-relational data. Bordes et al., “Translating embeddings for modeling multi-relational data,” In Advances in Neural Information Processing Systems. 2787-2795 (2013) (“TransE”).

Translated embeddings can then be clustered and/or analyzed with respect to the expression in which they originated and, based on the clustering/analysis, embedded into an (augmented) knowledge graph. In response to a query, such as a question requesting candidate therapeutics for a particular disease or biomarker entity, responses may be generated based on entities/relationships established within the (augmented) knowledge graph.

In some embodiments, the dissimilarity measure includes representing relationships using a binary representation and applying a scoring function and an inner dot product with complex components (e.g., Hermitian dot product). For example, methods are described for training entity relations and extracting new relations from existing ones in U.S. Patent Application Publication No. 20170337481 A1, entitled “Complex embeddings for simple link prediction” (“ComplEx”).

In some embodiments, among at least a portion of the entities and relationships used to train the machine learning model, at least one element of the entities is replaced with a random entity. For example, applying a loss function or scoring function described above can be integrated with a random forest (RF) model.

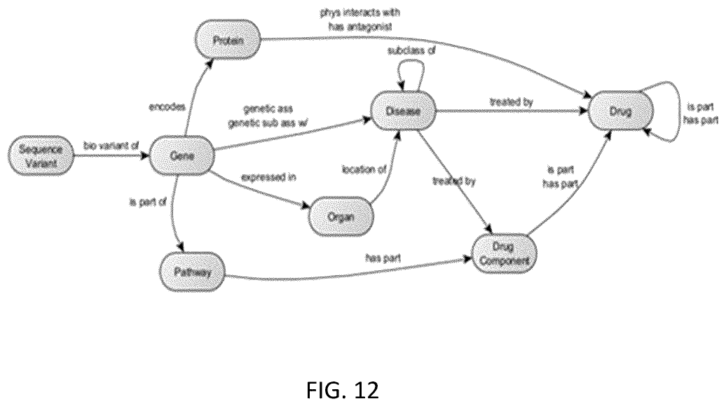

FIG. 12 is an illustrative flow diagram of processes for identifying a therapeutic candidate from a biomedical knowledge graph based on a particular sequence variant according to some embodiments. In response to a query regarding a particular sequence variant (e.g., obtained from a tissue/blood sample from a patient), patterns and relationships can be searched within the biomedical data and/or knowledge graph to identify new or existing relationships between the sequence variant (e.g., from a cancerous tumor in a particular organ) and a candidate therapeutic (e.g., immunotherapy, chemotherapy).

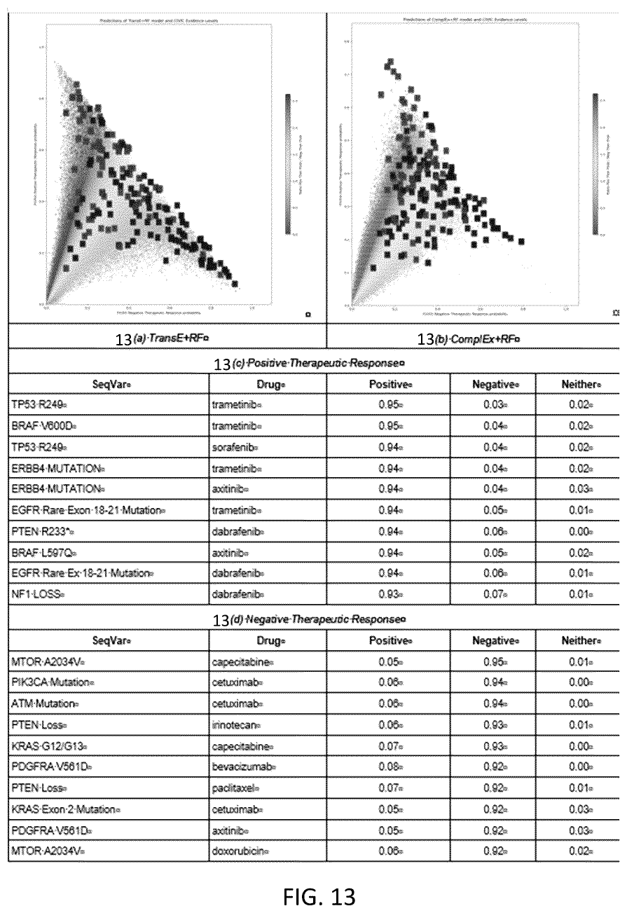

FIG. 13 is an exemplary output of candidate therapeutics with predicted response characteristics generated from a biomedical knowledge graph in relation to particular sequence variants according to some embodiments. As depicted in the scatter plot 13(a) where each dot represents a pair of ‘genetic variant-drug’ pair predicted by utilizing the “TransE” learning model combined with random forest (RF) (“TransE+RF”), most of the predictions are in areas where both (positive and negative therapeutic response probabilities) are similar so positive to negative ratios are close to 1.

We see groups of predictions with a very high probability of having a positive therapeutic response and a very low probability of having a negative therapeutic response (top-left corner of the scatter plot) and in the bottom-right corner a subgroup of predictions with a very high probability of having a negative therapeutic response but a very low probability of having a positive therapeutic response. Scatter plot 13(b) represents a pairs predicted by utilizing the “ComplEx” learning model combined with random forest (RF) (“ComplEx+RF”). Visual inspection of the plots suggest how the models have a fairly good agreement between the predictions and the annotations present in Clinical Interpretations of Variants in Cancer (CIVIC) even when the models have never been trained against them.

Table 13(c) of FIG. 13 illustrates ten top predictions obtained by the TransE+RF model, excluding the training set, with the highest predicted probability of having a “P3354 Positive Therapeutic Response” relationship. Interestingly three of the four drugs in the top 10 (trametinib, sorafenib and axitinib) are kinase inhibitors. Table 13(d) of FIG. 13 shows ten top triplets with a highest predicted probability of having a P3355 negative therapeutic response” relationship.

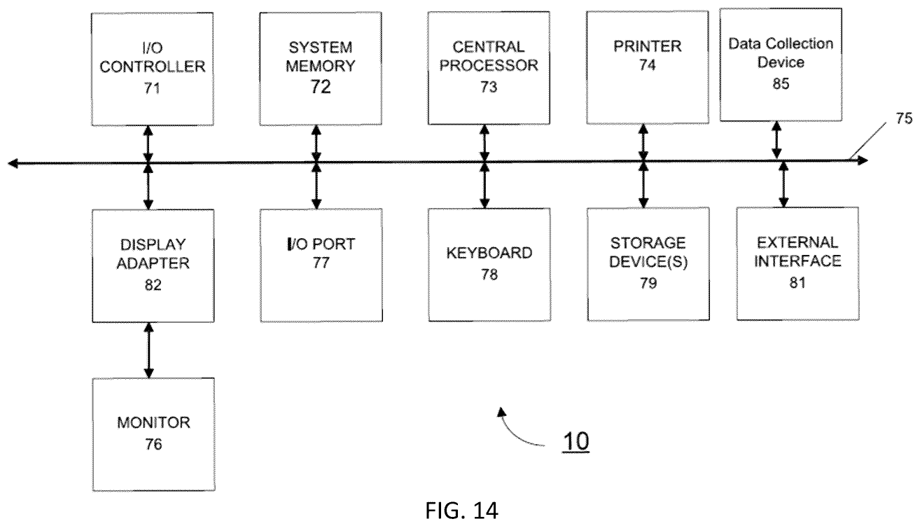

FIG. 14 is an illustrative diagram of computing devices and processing components of a system for generating and utilizing biomedical knowledge graphs according to some embodiments. Any of the computer systems mentioned herein may utilize any suitable number of subsystems. Examples of such subsystems are shown in FIG. 14 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices. In some embodiments, a cloud infrastructure (e.g., Amazon Web Services), a graphical processing unit (GPU), etc., can be used to implement the disclosed techniques.

The subsystems shown in FIG. 14 are interconnected via a system bus 75. Additional subsystems such as a printer 74, keyboard 78, storage device(s) 79, monitor 76, which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire®). For example, I/O port 77 or external interface 81 (e.g. Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81 or by an internal interface. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware (e.g. an application specific integrated circuit or field programmable gate array) and/or using computer software with a generally programmable processor in a modular or integrated manner. As used herein, a processor includes a single-core processor, multi-core processor on a same integrated chip, or multiple processing units on a single circuit board or networked. Based on the disclosure and teachings provided herein, a person of ordinary skill in the art will know and appreciate other ways and/or methods to implement embodiments of the present invention using hardware and a combination of hardware and software.

Any of the software components or functions described in this application may be implemented as software code to be executed by a processor using any suitable computer language such as, for example, Java, C, C++, C #, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective steps or a respective group of steps. Although presented as numbered steps, steps of methods herein can be performed at a same time or in a different order. Additionally, portions of these steps may be used with portions of other steps from other methods. Also, all or portions of a step may be optional. Additionally, any of the steps of any of the methods can be performed with modules, units, circuits, or other means for performing these steps.

The specific details of particular embodiments may be combined in any suitable manner without departing from the spirit and scope of embodiments of the invention. However, other embodiments of the invention may be directed to specific embodiments relating to each individual aspect, or specific combinations of these individual aspects.

The above description of example embodiments of the invention has been presented for the purposes of illustration and description. It is not intended to be exhaustive or to limit the invention to the precise form described, and many modifications and variations are possible in light of the teaching above. A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover reference to a “first” or a “second” component does not limit the referenced component to a particular location unless expressly stated.

The above-described embodiments of the present disclosure are presented for purposes of illustration and not of limitation, and the present disclosure is limited only by the claims which follow. Furthermore, it should be noted that the features and limitations described in any one embodiment may be applied to any other embodiment herein, and flowcharts or examples relating to one embodiment may be combined with any other embodiment in a suitable manner, done in different orders, or done in parallel. In addition, the systems and methods described herein may be performed in real time. It should also be noted, the systems and/or methods described above may be applied to, or used in accordance with, other systems and/or methods.

All patents, patent applications, publications, and descriptions mentioned herein are incorporated by reference in their entirety. None is admitted to be prior art.

1.-21. (canceled)

22. A computer-implemented method for structuring and retrieving biomedical data, the method comprising:
obtaining a knowledge graph of biomedical information representing a plurality of nodes and connections between the nodes, wherein each of the nodes represent a biomedical entity and each of the connections represent a biomedical relationship between the biomedical entities;
obtaining biomedical data from one or more data sources;
extracting, from the biomedical data, a plurality of biomedical entities and biomedical relationships between the entities;
identifying a plurality of entities and relationships according to a predefined schema for a biomedical knowledge graph, the identifying comprising:
entering the plurality of entities and relationships into a machine learning model, the model trained to identify relationships between entities extracted from the biomedical data based on established relationships within the biomedical knowledge graph, the identifications based on a dissimilarity measure between related entities;
determining biomedical entities, entity types, and entity relationships in the data by searching for predetermined identifiers or patterns in the data and based on an output from the machine learning model in response to entering the plurality of entities and relationships;
based on the determined biomedical type of the entity, assigning each biomedical entity to a cluster of biomedical entity types;
identifying a context for each of the identified biomedical entities based on the assigned cluster and based on elements of an expression of the biomedical data within which the entity is expressed;
based on the identified context and type of the biomedical entities, incorporating records of nodes and connections between nodes into the knowledge graph, the nodes representing biomedical entities and the connections representing biomedical relationships between the entities structured according to the predefined schema;
receiving a query for biomedical information;
converting the query into a structured query expression based on the predefined schema; and
generating a query result of biomedical information based on searching through the knowledge graph using the structured query expression.

23. The method of claim 22 wherein the training of the machine learning model comprises applying a margin-based ranking criterion using a margin hyperparameter over the established relationships within the biomedical knowledge graph, and applying a loss function to triplets extracted from the established relationships.

24. The method of claim 23 wherein the loss function comprises penalizing incorrect associations in the knowledge graph while maximizing or optimizing the margin between positive and negative triples.

25. The method of claim 24 wherein the machine learning model comprises a scoring function that computes a distance between entities and relationships between entities, minimizing the distance for incorrect relationships and maximizing the distance for incorrect relationships.

26. The method of claim 25 wherein the distance is calculated using at least one of a L1 or L2 norm.

27. The method of claim 23 wherein the training of the machine learning model comprises establishing relationships using a binary representation and applying a scoring function and
inner product using both real and imaginary spaces.

28. The method of claim 27 wherein the inner product comprises a Hermitian dot product.

29. The method of claim 22 wherein, among at least a portion of the entities and relationships, at least one element of the entities is replaced with a random entity.

30. The method of claim 22 wherein the one or more data sources comprises clinical trials data, journal publications, and/or a generalized knowledge graph.

31. The method of claim 22 wherein determining biomedical entities and entity types comprises utilizing a named entity resolution (NER) module.

32. The method of claim 22 wherein identifying a context comprises utilizing a natural language processing (NLP) module.

33. The method of claim 22 wherein the schema comprises entities classified as one of a gene, sequence, anatomic structure, chemical substance, disease, and phenotypic feature.

34. The method of claim 22 wherein the schema comprises relationship types among entities classified as at least one of positively regulating or negatively regulating biological factors.

35. The method of claim 22 wherein the context comprises at least one of a gene sequence, an identification of a biomarker associated with a disease, or a hypothesis within a medical publication.

36. The method of claim 31 wherein the relationships comprise at least one an action via a receptor, indirect alteration of an effect of an endogenous agonist, an inhibition of transport processes, enzyme inhibition, enzymatic action or activation of enzymatic activity, chelation, osmosis, and/or anesthesia.

37. The method of claim 22 further comprising predicting and reporting biomedical relationships among the biomedical entities using a machine learning model that receives data from the biomedical knowledge graph as input.

38. The method of claim 22 wherein converting the query into a structured query expression comprises utilizing a machine learning based NLP model optimized for structuring the query for searching the biomedical knowledge graph with the predefined schema.

39. A biomedical knowledge graph system, the system comprising:
a computer database of records, the records comprising nodes of biomedical entities and connections between the entities representing biomedical relationships;
one or more processors programmed and configured to:
obtain a knowledge graph of biomedical information representing a plurality of nodes and connections between the nodes, wherein each of the nodes represent a biomedical entity and each of the connections represent a biomedical relationship between the biomedical entities;
extract data from a plurality of data sources;
entering the plurality of entities and relationships into a machine learning model, the model trained to identify relationships between entities extracted from the biomedical data based on established relationships within the biomedical knowledge graph, the identifications based on a dissimilarity measure between related entities;
determine biomedical entities, entity types, and entity relationships in the data by searching for predetermined identifiers or patterns in the data and based on an output from the machine learning model in response to entering the plurality of entities and relationships;
based on the determined biomedical entities, assign each biomedical entity to a cluster of biomedical entity types;
identify a context for each of the identified biomedical entities based on the assigned cluster and based on elements of the expression of the biomedical data within which the entity is expressed; and
based on the identified context and type of the biomedical entity, incorporating records of nodes and connections between nodes into the knowledge graph, the nodes representing biomedical entities and the connections representing biomedical relationships between the entities structured according to a predefined schema.

40. The system of claim 39 wherein the training of the machine learning model comprises applying a margin-based ranking criterion using a margin hyperparameter over the established relationships within the biomedical knowledge graph, and applying a loss function to triplets extracted from the established relationships.

41. The system of claim 40 wherein the loss function comprises penalizing incorrect associations in the knowledge graph while maximizing or optimizing the margin between positive and negative triples.

42.-52. (canceled)
